(19) United States
(12) Patent Application Publication
(10) Pub. No.: US 2013/0250078 A1
(43) Pub. Date: Sep. 26, 2013

(54) VISUAL AID

(71) Applicant: TECHNOLOGY DYNAMICS INC., Bergenfield, NJ (US)

(72) Inventor: Aron LEVY, Bergenfield, NJ (US)

(73) Assignee: Technology Dynamics Inc., Bergenfield, NJ (US)

(21) Appl. No.: 13/770,560

(22) Filed: Feb. 19, 2013

Related U.S. Application Data
(60) Provisional application No. 61/615,401, filed on Mar. 26, 2012.

(51) Int. Cl. A61F 9/08

(52) U.S. Cl. CPC A61F 9/08
USPC 348/62

(57) ABSTRACT

A visual aid system, device and method is provided. The visual aid system may include an imaging unit to capture images of a user's surroundings, a knowledge database to store object recognition information for a plurality of image objects, an object recognition module to match and identify an object imaged in one or more captured images with an object in the knowledge database, and an output device to output a non-visual indication of the identified object.

### CROSS-REFERENCE TO RELATED APPLICATIONS

This application claims the benefit of prior U.S. Provisional Application Ser. No. 61/615,401, filed Mar. 26, 2012, which is incorporated by reference herein in its entirety.

### FIELD OF THE INVENTION

Embodiments of the present invention relate to visual aid systems, devices and methods, for example, to aid visually impaired or blind users.

### BACKGROUND OF THE INVENTION

Visually impaired and blind people currently rely on dogs or canes to detect obstacles and move forward safely. Recent advances include a device referred to as a “virtual cane” that uses sonar technology to detect obstacles by transmission and reception of sonic waves.

However, these solutions only detect the presence of an obstruction and are simply tools to avoid collision. They cannot identify the actual object that causes the obstruction, for example, distinguishing between a chair and a pole, or provide a spatial view of the landscape in front of the user.

There is a long felt need in the art to provide visually impaired users with an understanding of their environment that mimics the visual sense.

### SUMMARY OF THE INVENTION

Embodiments of the invention may provide a visual aid system, device and method. The visual aid system may include an imaging unit to capture images of a user's surroundings, a knowledge database to store object recognition information for a plurality of image objects, an object recognition module to match and identify an object in one or more captured images with an object in the knowledge database, and an output device to output a non-visual indication of the identified object.

### BRIEF DESCRIPTION OF THE DRAWINGS

The principles and operation of the system, apparatus, and method according to embodiments of the present invention may be better understood with reference to the drawings, and the following description, it being understood that these drawings are given for illustrative purposes only and are not meant to be limiting.

FIG. 1 is a schematic illustration of a visual aid system in accordance with embodiments of the invention; and

FIG. 2 is a flowchart of a method for using the visual aid system of FIG. 1 in accordance with embodiments of the invention.

For simplicity and clarity of illustration, elements shown in the drawings have not necessarily been drawn to scale. For example, the dimensions of some of the elements may be exaggerated relative to other elements for clarity.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Embodiments of the present invention allow blind or visually impaired users to “see” with their other senses, for example, by hearing an oral description of their visual environment or by feeling a tactile stimulus. Embodiments of the present invention may include an imaging system to image a user's surroundings in real-time, an object recognition module to automatically recognize and identify visual objects in the user's path, and an output device to notify the user of those visual objects using non-visual (e.g., oral or tactile) descriptions to aid the visually impaired user. The object recognition module may access a knowledge database of stored image objects to compare to the captured image objects and, upon detecting a match, may identify the captured object as its matched database counterpart. Each matched database object may be associated with a data file of a non-visual description of the object, such as an audio file of a voice stating the name and features of the object or a tactile stimulus. The output device may output or play the non-visual description to the visually impaired user.

Such embodiments may report visual objects, people, movement and scenery in the user's field of view using non-visual descriptors. A user may use such systems, for example, to recognize people, places and objects while they walk outside, to find the correct drug label when they open a medicine cabinet, to find the correct street address from among a row of houses or businesses, to avoid collisions, etc. Embodiments of the invention may not only be a tool to avoid obstacles, but may actually mimic the sense of sight including both the functionality of the eyes (e.g., using a camera) and the visual recognition and cognition pathways in the brain (e.g., using an object recognition module).

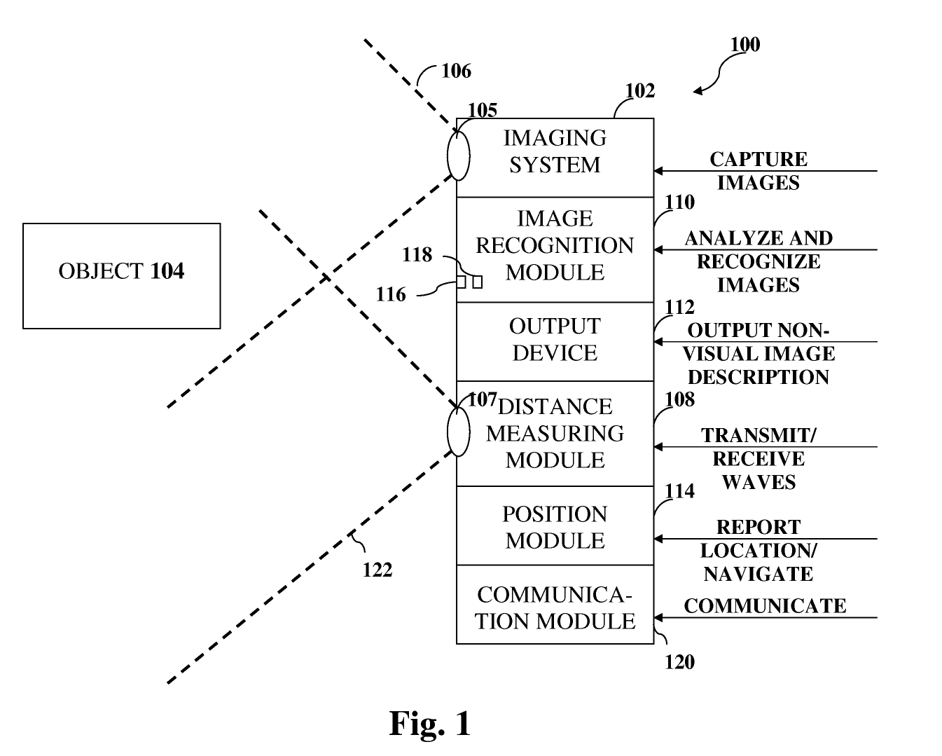

Reference is made to FIG. 1, which schematically illustrates a visual aid system 100 in accordance with embodiments of the invention.

System 100 may include an imaging system 102 to image light reflected from objects 104 in a field of view (FOV) 106 of imaging system 102. System 100 may include a distance measuring module 108 to measure the distance between module 108 and object 104, an object recognition module 110 to identify images of object 104, an output device 112 to output a non-visual indication of the identity of object 104 and a position module 114 to determine the position of system 100 or object 104. System 100 may include a transmitter or other communication module 120 to communicate with other devices, e.g., via a wireless network. System 100 may optionally include a recorder to record data gathered by the device, such as, the captured image data.

Imaging system 102 may include an imager or camera 105 and an optical system including one or more lens(es), prisms, or mirrors, etc. to capture images of physical objects 104 via the reflection of light waves therefrom in the imager' s field of view 106. Camera 105 may capture individual images or a stream of images in rapid succession to generate a movie or moving image stream. Camera 105 may include, for example, a micro-camera, such as, a “camera on a chip” imager, a charge-coupled device (CCD) and/or metal-oxide-semiconductor (CMOS) camera. The captured image data may be digital color image data, although other image formats may be used. Camera 105 may be worn by the user, such as, on a hat, a belt, the bridge of a pair of glasses or sunglasses, an accessory worn around the neck to suspend camera 105 near chest level, or worn near ground level attached to shoe laces or the tongue of a shoe. The camera's field of view 106 may be similar to that of the human eye system (e.g., approximately 160° in the horizontal direction and 140° in the vertical direction) or may be wider (e.g., approximately 180° or 360°) or narrower (e.g., approximately 90°) in the vertical and/or horizontal directions. In some embodiments, camera 105 may move or rotate to scan its surroundings for a dynamic field of view 106. Scanning may be initiated automatically or upon detecting a predetermined trigger, such as, a moving object. In some embodiments, a single camera 105 may be used, while in other embodiments, multiple cameras 105 may be used, for example, to assemble a panoramic view from the individual cameras. Imaging system 102 may capture images periodically. The periodicity may be set and/or adjusted by the programmer or user to be a predetermined time, for example, either in relatively fast succession (e.g., 10-100 frames per second (fps)) to resemble a movie or in relatively slow succession (e.g., 0.1-1 frames per second) to resemble individual images. In other embodiments, imaging system 102 may capture images in response to a stimulus, such as, a change in visual background, change in light levels, rapid motion, etc. Imaging system 102 may capture images in real-time.

Object recognition module 110 may analyze the image data collected by imaging system 102. Object recognition module 110 may include a processor 118 to execute object recognition logic including, for example, image recognition, pattern recognition, spatial perception, motion analysis and/or artificial intelligence (AI) functionalities. The logic may be used to compare features of the collected image data to known images or object recognition information stored in an image dictionary or knowledge database, e.g., located in a memory 116 or an external database. For example, object recognition module 110 may identify and extract a main, moving or new object in an image and compare it to known image objects represented in the knowledge database. Object recognition module 110 may compare the captured extracted object and dictionary objects using the actual images of the objects, metadata derived from the images or annotated or summary information associated with the images. In some embodiments, object recognition module 110 may compare images based on one or more features of the imaged object 104, such as, object name (e.g., an apple vs. a hammer), object type or category (e.g., plant vs. tool), color, size, shape, texture, pattern, brightness, distance to the object and direction or orientation of the object. Each feature may be determined using a separate comparison. The knowledge database may also store a data file of a non-visual description of each database object and/or feature, such as an audio file reciting the name of the object and its associated features, a tactile stimulus defining the presence of a new object or a near object likely to cause a collision, etc. Accordingly, when a match is found between the imaged object and database object, output device 112 may output or play the associated non-visual description to the visually impaired user for recognition of his/her surroundings. Output device 112 may include headphones, speakers, etc., to output sound data files and a buzzer, micro-electromechanical systems (MEMS) switch or vibrator to output tactile stimuli.

In some embodiment, the knowledge database may be adaptive. An adaptive knowledge database may store object recognition information, not only for generic objects, like apple or chair, but also individualized objects for user-specific recognition capabilities. The adaptive knowledge database may be used, for example, to recognize a user's family, friend and co-workers identifying each individual by name, to recognize the streets in the user's town, the office where the user works, etc. A machine-learning or “training” mode may be used to add objects into the knowledge database, for example, where the user may put the target object into field of view 106 of camera 105 and input (e.g., type or speak) the name or features of the new imaged object. In other embodiments, knowledge database may be self-adaptive or self-taught. In one example, when an unknown object commonly appears in the user's path, object recognition module 110 may automatically access a secondary knowledge database, e.g., via communication module 120, to find the recognition information associated with that object and add it to the primary knowledge database.

Distance measuring module 108 may measure the distance between module 108 and object 104. Distance measuring module 108 may include a transmitter/receiver 107 to transmit waves, such as, sonar, ultrasonic, and/or laser waves, and receive the reflections of those waves off of object 104 (and noise from other objects) to gauge the distance to object 104. Distance measuring module 108 may emit waves in a range 122 by scanning an area, for example, approximating field of view 106. The received wave information may be input into a microcontroller (e.g., in module 108) programmed to identify the distance to object 104. The distance measurement may be used for collision avoidance to alert the user with an alarm (e.g., an auditory or tactile stimulus) via output device 112 when a possible collision with object 104 is detected. A possible collision may be detected when the distance measured to object 104 is less than or equal to a predetermined threshold and/or when the user and/or object 104 is moving. Distance measuring module 108 may alert the user to avoid objects 104 (still or moving) which are pre-identified as threatening and/or may recommend the user to halt or change course (e.g., “turn left to avoid couch”). The distance measurement may also be used for size calculations, for example, scaling the size of object 104 in the image by a factor of the distance measured, to determine the actual size of object 104, for example, to describe object 104 as “large,” “medium” or “small” relative to a predefined standard size of the object.

A position module 114 may include a global positioning system (GPS), accelerometer, compass, gyroscope etc., to determine the position, speed, orientation or other motion parameters of system 100 and/or object 104. Position module 114 may report a current location to the user and/or guide the user as a navigator device. For example, position module 114 may provide oral navigation directions responsive to avoid obstructive objects identified, for example, by object recognition module 110 using the captured images and/or by distance measuring module 108 using wave reflection data, for real-time guidance adaptive to the user's environment. For example, if object recognition module 110 detects an obstruction in a navigational path proposed by position module 114, such as a closed road or a pot hole, position module 114 may re-route the navigational path around the obstruction.

Communication module 120 may include a transmitter and receiver to allow system 100 to communicate with remote servers or databases over networks, such as the Internet, e.g., via wireless connection. Communication module 120, in conjunction with position module 114, may allow a remote server to track the user, communicate with the user via output device 112, and send information to the user, such as, auditory reports of street closings, risky situations, the news or the weather.

Components of system 100 may have artificial intelligence logic installed, for example, to fully interact with the user based on non-visual queues, for example, by accepting and responding to vocal commands, vocal inquiries and other voice activated triggers. For example, the user may state a command, e.g., via a microphone or other input device, causing camera 105 to scan its surroundings or position module 116 to navigate the user to a requested destination.

System 100 components may communicate with each other and/or external units via wired or wireless connections, such as, Bluetooth or the Internet. Components of system 100 may be integrated into a single unit (e.g., all-in-one) or may include multiple separate pieces or sub-units. One example of an integrated system 100 may include glasses or sunglasses in which camera 105 is placed at the nose bridge and/or earphone output devices 112 extend from the temple arms. Micro or lightweight components may be used for the comfort of the glasses system 100. Another example of an integrated system 100 may include a headphone output device 112 with camera 105 attached at the top of the headphone bridge.

System 100 may be configured to exclude some components, such as, communication module 120, or include additional components, such as, a recorder. Other system 100 designs may be used.

Embodiments of the present invention may describe visual aspects of the user's environment using non-visual descriptions, triggers or alarms. For example, sensory input related to a first sense (e.g., visual stimulus) may be translated or mapped to sensory information related to a second sense (e.g., auditory or tactile stimulus), for example, when the first sense is impaired. Such embodiments may convey details or features of the sensed objects, such as, the color or shape of the object extending beyond the capabilities of current collision avoidance mechanisms. Embodiments of the invention may use artificial intelligence to interpret images in the user's field of view 106, similarly to the human visual recognition process, and to provide such information orally to the visually impaired user. Such description may provide insights and detail beyond what visually impaired user can recognize simply by feeling objects around them or listening to ambient sounds. Such visual descriptions may evoke memory and visual queues present for users who previously had a functioning sense of sight. For example, auditory descriptions of visual objects may activate regions of the brain, such as the occipital lobe, designated for visual function, even without the function of the eyes. Embodiments of the invention may allow users to “visualize” the world through an orally description of the images captured by imaging system 102.

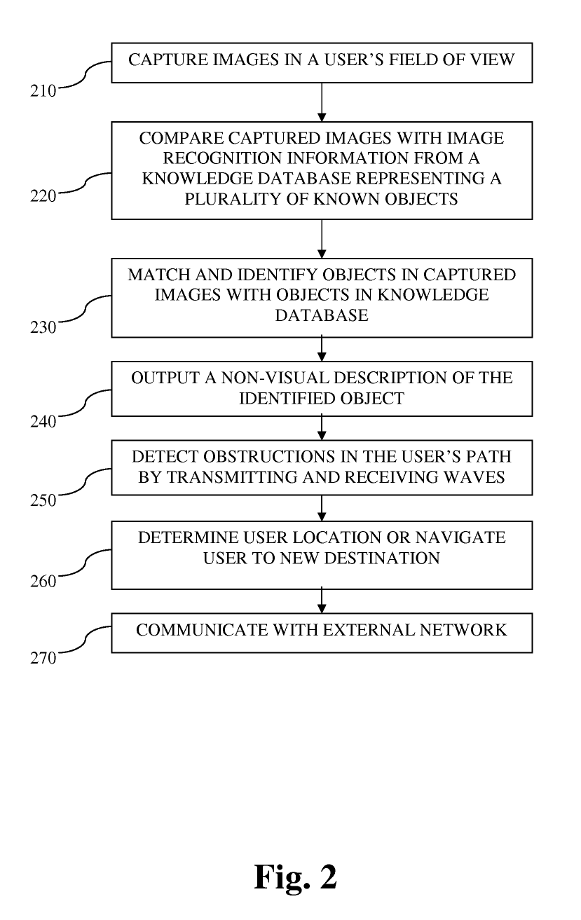

Reference is made to FIG. 2, which is a flowchart of a method for using a visual aid system (e.g., system 100 of FIG. 1) in accordance with embodiments of the invention.

In operation 210, an imaging system (e.g., imaging system 102 of FIG. 1) may capture images in the user's surroundings or field of view (e.g., field of view 106 of FIG. 1).

In operation 220, an object recognition module (e.g., object recognition module 110 of FIG. 1) may compare the images captured in operation 210 with images or associated object recognition information for predefined objects stored in a knowledge database (e.g., memory 116 of FIG. 1).

In operation 230, the object recognition module may match the captured image objects (e.g., objects 104 of FIG. 1) with image objects represented in the knowledge database. When a match is found, the captured image object may be identified or recognized as the predefined database object.

In operation 240, an output device (e.g., output device 112 of FIG. 1) may output or play a non-visual descriptive file associated with the matching database object (e.g., a sound file or command to activate a tactile stimulus device).

In operation 250, a distance measuring module (e.g., distance measuring module 108 of FIG. 1) may measure the distance between the imaging system and the imaged object. In one example, the distance measuring module may emit and receive waves to gauge the distance of the reflected wave path to/from the object.

In operation 260, a position module (e.g., position module 114 of FIG. 1) may determine the position or motion parameters of the user and/or the imaged object.

In operation 270, a communication module (e.g., communication module 120 of FIG. 1) may allow the user and the system components to communicate with other external devices.

Other operations or orders of operations may be used.

When used herein, “visually impaired” may refer to a full or partial loss of sight in humans (partially sighted, low vision, legally blind, totally blind, etc.) or may refer to users for whose visual field is obstructed, e.g., from viewing the rear or periphery in a plane or car, but who otherwise have acceptable vision. Furthermore, embodiments of the invention may be used in other contexts when vision is not an issue, for example, for identifying individuals in a diplomat meeting, identifying landmark structures as a tourist, identifying works of art in a museum, for teaching object recognition to children, etc. In one example, a soldier or a policeman may use the device in situations where they may be attacked from behind. Their device, e.g., worn on the back of a helmet or vest, may scan a field of view behind them and alert them orally of danger, thus allowing them to remain visually focused on events in front of them. In another example, an imaging system (e.g., imaging system 102 of FIG. 1) may include a probe or robot (e.g., detached from the user) that may enter areas restricted to humans, such as, dangerous areas during a war, areas with chemical or biological leaks, extra-planetary space missions, etc. If the operation is executed in darkness, the imaging system may use night-vision technology to detect objects, where the user (e.g., located remotely) may request oral descriptions of the images due to the darkness. In another example, if the camera is equipped with night-vision, a user may be able to use it to visualize dimly lit streets or other dark places.

Although embodiments of the invention are described herein to translate visual sensory input for sight to auditory sensory input for hearing, such embodiments may be generalized to translate sensory input from any first sense to any second sense, for example, when the first sense is impaired. For example, sound input may be translated to visual stimulus, for example, to aid deaf or hearing impaired people. In another example, a tactile stimulus may be used to convey the visual and/or auditory world to a blind and/or deaf person.

It may be noted that robotics object recognition maps visual, auditory and all other sensory input to non-sensory data since robots, unlike humans, do not have senses. Accordingly, the object recognition systems of robotics networks would not be modified to transcribe visual sensory data into auditory data, since the auditory output would be inoperable in commanding or communicating with a robot.

It may be appreciated that capturing images and recognizing and reporting imaged objects in “real-time” may refer to operations that occur instantly, at a small time delay of, for example, between 0.01 and 10 seconds, while the object is in front of the viewer, etc.

Different embodiments are disclosed herein. Features of certain embodiments may be combined with features of other embodiments; thus certain embodiments may be combinations of features of multiple embodiments.

Embodiments of the invention may include an article such as a computer or processor readable non-transitory storage medium, such as for example a memory, a disk drive, or a USB flash memory encoding, including or storing instructions, e.g., computer-executable instructions, which when executed by a processor or controller (e.g., such as processor 118 of FIG. 1), cause the processor or controller to carry out methods disclosed herein.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. A system comprising:
an imaging unit to capture images of a user's surroundings;
a knowledge database storing object recognition information for a plurality of image objects;
an object recognition module to match and identify an object in one or more captured images with an object in the knowledge database; and
an output device to output a non-visual indication of the identified object.

2. The system of claim 1, wherein the non-visual indication is an audio file reciting the name of the object.

3. The system of claim 1, wherein the non-visual indication is a tactile stimulus.

4. The system of claim 1, further comprising a collision avoidance module to detect obstructions by transmitting and receiving of waves.

5. The system of claim 4, further comprising a positioning system to navigate the user using non-visual indications of directions that is responsive to avoid obstructive objects identified in the captured images or in the reflection of transmitted waves.

6. The system of claim 1, wherein the knowledge database is adaptive enabling new image recognition information to be added to the knowledge database for recognizing new objects.

7. The system of claim 1, wherein the object in the captured images is matched to multiple objects in the knowledge database, each knowledge database object associated with a different feature of the captured image object.

8. The system of claim 7, wherein the features are selected from the group consisting of: object name, object type, color, size, shape, texture, pattern, brightness, distance to the object, direction to the object and orientation of the object.

9. The system of claim 7 comprising a plurality of modes for object recognition selected from the group consisting of: standard mode indicating one or more features identified for each new object, quiet mode indicating only the object type feature for each new object, motion mode indicating new objects only when the environment changes, emergency mode indicating objects only when a collision is anticipated, scan mode identifying a plurality of objects in a current environment.

10. A method comprising:
capturing images of a user's surroundings;
storing object recognition information for a plurality of image objects;
identifying an object in one or more captured images that matches an object in the knowledge database; and
outputting a non-visual indication of the identified object.

11. The method of claim 10, wherein the non-visual indication is an audio file reciting the name of the object.

12. The method of claim 10, wherein the non-visual indication is a tactile stimulus.

13. The method of claim 10, further comprising detecting obstructions by transmitting and receiving of waves.

14. The method of claim 13, further comprising navigating the user using non-visual indications of directions that is responsive to avoid obstructive objects identified in the captured images or in the reflection of transmitted waves.

15. The method of claim 10 comprising adapting the knowledge database by enabling new image recognition information to be added to the knowledge database for recognizing new objects.

16. The method of claim 10 comprising matching the object in the captured images to multiple objects in the knowledge database, each knowledge database object associated with a different feature of the captured image object.

17. The method of claim 16, wherein the features are selected from the group consisting of: object name, object type, color, size, shape, texture, pattern, brightness, distance to the object, direction to the object and orientation of the object.

18. The method of claim 16 comprising operating according to a selected one of a plurality of modes for object recognition selected from the group consisting of: standard mode indicating one or more features identified for each new object, quiet mode indicating only the object type feature for each new object, motion mode indicating new objects only when the environment changes, emergency mode indicating objects only when a collision is anticipated, scan mode identifying a plurality of objects in a current environment.
